(19) United States
(12) Patent Application Publication
(10) Pub. No.: US 2014/0088570 A1
(43) Pub. Date: Mar. 27, 2014

(54) MEDICAL DEVICE PROVIDED WITH AN INNOVATIVE MARKING SYSTEM ENABLING RAPID, MULTIPLE AND REMOTE IDENTIFICATION

(71) Applicant: Marc Antoine Sergeant, Douai (FR)

(72) Inventor: Marc Antoine Sergeant, Douai (FR)

(21) Appl. No.: 14/113,848

(22) PCT Filed: Apr. 24, 2012
(86) PCT No.: PCT/FR2012/050898

§ 371 Date: Dec. 10, 2013

(30) Foreign Application Priority Data
Apr. 26, 2011 (FR) 1101311

(51) Int. Cl. A61B 19/00

(52) U.S. Cl. CPC A61B 19/44; A61B 19/54
USPC 606/1

(57) ABSTRACT

A medical device includes a reusable or non-reusable surgical instrument made from metal, polymer or plastic, or any implant or prosthesis substitute made from metal, polymer or plastic, which is equipped with a marking system enabling rapid identification of the instrument. The marking system includes: an RFID component; and a resin protuberance which is rigidly connected to the surgical instrument or implant substitute to be marked, the protuberance containing, and completely surrounding the tag.

An aspect of the present invention relates to the field of the remote identification of reusable medical equipment. The present invention proposes more particularly a system for integrating an identification component, of RFID component type, in a resin protuberance, on a reusable surgical instrument made from metal, polymer or plastic. This present invention could also apply to implant or prostheses substitutes made from metal, plastic or polymer.

The conclusion is simple: the number of deaths linked to nosocomial infections has remained high over several years, the different current measures are not sufficient to reduce this rate in a radical manner, in addition with a view to reducing costs, health establishments, whether private or public, are seeking means to optimise their management. The sterilisation of health establishments, places where the instruments are cleaned then sterilised, as well as operating theatres, are areas where important progress can be made. Those skilled in the art know that an efficient solution, enabling a reduction in costs but also enabling a better management of risks, is traceability to the surgical instruments. Mention will be made herein of surgical instruments, but everything that is mentioned could also apply to implant and prosthesis substitutes as well as any reusable medical equipment, where traceability to the element is worthwhile.

Those skilled in the art know that a simple traceability making it possible to identify remotely an instrument, save and update any useful information relating to the instrument, for example, its serial number, the clinical history of the patients operated, the specifications for use, maintenance, and sterilisation, but also to identify remotely several instruments, has benefices from a financial point of view, but would also facilitate the management of risks. It makes it possible firstly to give a characteristic name to an instrument, as well as a purchase date, which makes it possible to bring into play the manufacturer's guarantees, an action that cannot be carried out without identification of the instrument.

In addition, a recent study has revealed that more than one operating incident out of three is due to poor management of surgical instruments, this being due firstly to the stress of the surgeons and nurses usually caused through poor management of surgical instruments. By ensuring traceability to the instrument, it makes it possible to acquire the certitude that a box of instruments is complete. Thus, it is known directly if all of the instruments at the entrance to the operating theatre are present in the box. In fact, recurring problems are incomplete or incorrectly constituted boxes.

The number of nosocomial infections contracted in operating theatres is non-negligible. A good means for reducing this risk, in addition to the conventional constraints on health safety in operating theatres (white areas), is to trace the instrument. With traceability, one can be sure that the instrument will have indeed followed the sterilisation steps. In addition, this technology makes it possible to certify that all of the marked instruments have indeed followed the complete sterilisation process, and also not to mix up single use instruments with reusable instruments. Numerous cases of fraud have been detected in hospitals, which could lead to an increase in the risk of contamination of patients. Recent cases, such as that of the Clinique du Sport, have shown the importance of strictness in the sterilisation cycles of the surgical instruments used. In this case, more than 1300 patients were concerned by the bacterium Xenopi. Such a fraud would not have been possible in the case where the instruments had been identified and certified by traceability.

On a legal level, several regulations have been introduced since 2001 concerning the traceability of instruments in France. In 2001 the circular DGS/5 C/DHOS/E 2 n° 2001-138 of the 14 Mar. 2001 relates to the precautions to observe while providing care to reduce the risks of transmission of non-conventional transmissible agents. In this circular, a first mention appears in relation to the traceability of instruments specifying that the establishment must do all in its power to facilitate the traceability of surgical instrument in the event of a problem. Other circulars followed, to arrive in 2009 at the WHO “Check-list” which imposes counting instruments before and after operations, yet there can be up to 500 instruments per operation, which creates an obvious loss of time. Traceability to the instrument would facilitate this step, and above all avoid human errors which can here be important.

Hospitals are criminally responsible for their acts. As an example, the AP-HP receives 1,200 annual complaints concerning medical errors or poor case management of patients as well as infections within the hospital. A better traceability of surgical instruments is the best means for hospitals to protect themselves against certain legal proceedings.

The legislation on prion imposes being capable of recognising the last patients operated with a medical device used on the patient in which an infectious risk has been discovered. Such recognition is only possible if it is possible to trace instruments one by one.

An incomplete or incorrect box arriving at the operating theatre constitutes a real loss of time. It is immediately sent back to the sterilisation area. Consequently, the instruments present in the box are thus again subjected to the whole sterilisation process. Traceability to the instrument makes it possible to know with certainty the presence of instruments in a box, whether at the sterilisation exit or at the entrance of the operating theatre. This thus avoids the presence of non-compliant boxes at the entrance of the operating theatre, and thus avoids loss of time in the planning of operations, and also reduces significantly the number of sterilisation cycles carried out needlessly.

The management of human resources and the allocation of their tasks remains a major problem in hospital structures. Yet, the constitution of the boxes, their preparation in the operating theatre, etc. constitute responsibilities of operating theatre nurses, functions that are currently lacking personnel. Identification at the instrument would make it possible to free the nurses from these time-consuming operations to delegate them to sterilisation operators who would only have to present the instruments in front of a detector, the software indicating to them the box corresponding to each of the instruments.

This traceability to the instrument provides a precious aid to the maintenance, the management of stocks and out of date items in the instrumentation inventory and for recomposing instrument trays for operating theatres during their retreatment.

Several systems of traceability to the instrument are found in the prior art. Firstly it may be observed that, in a certain number of establishments, the identification takes place visually, a type of instrument is recognised, and not an instrument in particular, by comparing it to a photo, in addition in compliance with the law the instruments are counted on entering and exiting the operating theatre. Since this process is uniquely visual, errors are numerous, and certain financial optimisation actions are impossible: the carrying out of a complete inventory, the management of the renewal and the maintenance of the pool of instruments, combatting theft or the loss of instruments, the reduction in the number of needless sterilisation cycles, or instead the management of human resources.

Another solution consists in carrying out a coloured marking of surgical instruments, particularly described in the patent application FR2760630A1, in this document it is provided to machine, at one or more places, the surgical instrument to append thereto coloured markers. By creating colour combinations, the operator can identify the instruments and thereby sort them in their respective boxes. This invention, despite an benefice of reduced cost, is very limited and has several weaknesses. In fact, since the number of instruments is high, the number of colour combinations also has to be high, an operator thus will not be able to memorize them, and he will be obliged to refer to a document making him lose precious time with regard to the quantity of instruments to be sorted out. Still linked to the increasing number of instruments and to the multiple combinations of colours that ensues therefrom, the instrument has to be machined at several places, which weakens the structure of the instrument and will increase the risk of loss of the colour marker, for example in the body of a patient. In addition, this solution does not have any financial benefice, it does not in any way enable an automation of part of the identification, or management, time or safety saving.

European patent number EP0681252A1, proposes a solution of traceability to the instrument belonging to identification by bar code, this technology is more commonly called “datamatrix” or two-dimensional bar code. This solution proposes marking surgical instruments by laser, by engraving on a part of the instrument a two-dimensional bar code, the code containing a certain number of information items. As for the conventional bar code, the identification has to take place at short distance by visual detection. Any opaque element located between the reader and the marked part of the instrument prevents the reading of the code. The interest of this technique is of being inexpensive, and more effective from a time saving point of view, than colour marking solutions. Nevertheless, several major imperfections go against the democratisation of this method: the presence of water between the bar code and the detector makes reading impossible, or in sterilisation the instruments are either humid, or dirty, in these conditions the operator has to find the spot where the bar code is located then wash this area and dry it before reading the code. Datamatrix does not enable any time saving, nor multiple or remote identification. The engraving of the instrument also has numerous drawbacks. Two possibilities exist: deeply marking the instrument with a laser or forming a superficial bar code. In the first case, the marking creates an important asperity at the surface of the instrument and constitutes a non-negligible area of risk with regard to the deposition of infectious elements, while weakening the structure of the instrument. In the second case, the marking easily disappears and needs to be reiterated often.

A more interesting solution is disclosed in French patent number FR2935602A1, as well as under similar forms in U.S. Pat. No. 7,837,684B2, French patent FR2947713A1, or European patent EP2116208A1, this solution advocates machining the surgical instrument to introduce therein a remote identification tag, better known under the name of RFID (Radio-frequency-identification) tag. Radio identification, usually designated by the acronym RFID (Radio Frequency Identification), is a method for memorizing and recovering data remotely using markers known as “radio-labels”, “RFID tags” or “RFID transponders”. The aim of this solution is to hollow out on the instrument a housing intended to receive inside it the transponder, or RFID tag, and to enclose it either by means of a material identical to the composition of the instrument, or by means of an element enabling radio waves to pass. This technology has non-negligible interets, such as the robustness of the support to the environment, the capacity for storing information items inside the chip, the facility of reading in inappropriate environments. Traceability to the instrument is only interesting when it confirms the following properties: rapid individual identification of instruments, inexpensive with regard to the unit cost of an instrument and complying with the health safety imperatives present in the hospital environment. Within the scope of the solution described in the aforementioned patents, machining each instrument and integrating therein tags of small size represents a considerable working time and proves to be extremely costly. In addition, it leads to a non-negligible weakening of the instrument. The efficiency of the action of the RFID is also reduced: the integration of a high frequency tag in a metal cavity leads to perturbations, not just in transmissions but also in the reception of electromagnetic waves, due to the known Faraday cage phenomenon in the case of metal instruments. The performances of the system are largely altered, reduced reading distance on contact and use of more costly detection equipment. A solution making it possible to overcome the problem of perturbation of the waves by the instrument could be to use low frequency RFID tags. Nevertheless, the performances of the overall system will not be improved, the detection of the instruments would then have to be carried out individually and the reading time would be substantially lengthened due to the periods of switching on and switching off the circuit of the chip of the RFID tag. In addition, in order to use this technology, the necessary detectors have to emit a concentric magnetic field, imposing a reading distance on contact and very high investments.

A final solution, very similar to that described previously, is proposed by the French firm IS-Médical in its patent application number FR2868393A1 dated the 14 Apr. 4. The firm also proposes integrating RFID tags in surgical instruments but here it advocates enclosing the tag in a metal capsule and welding this capsule to the instrument. Within the scope of this solution advantages exist: the robustness of the association between the RFID tag and the surgical instrument, the reliability of the link between the support and the instrument, the important information storage capacity of the chip, and the possibility of using a degraded environment, but the financial and technical constraints of weakening the instrument remain unchanged. This case comes close to that cited previously, the integration of an RFID tag within a metal cavity implies the obligation of using low frequency RFID tags. Also, technical tests have shown that the use of a concentric field of the transmitter is necessary, and consequently also imposes a reading on contact and individual of the instruments. In addition, as previously, the use of chips of small size, and the method of integration lead to costs, although less than the preceding solution, which still remain high.

The aim of the present invention is to overcome some of the drawbacks of the prior art, it proposes enabling traceability of a reusable or non-reusable surgical instrument, made from metal, polymer or plastic, by means of a reading system; this present invention could also apply to implant or prosthesis substitutes made from metal, plastic or polymer, dental or veterinary instruments. In the remainder of the document “element to be marked” designates any type of reusable or non-reusable surgical instrument made from metal, polymer or plastic, implant or prosthesis substitutes made from metal, polymer or plastic, dental or veterinary instruments.

The present invention aims to be able to respond to the aforementioned problems, problems encountered in sterilisation, and to overcome the limits of existing techniques. To this end, an aspect of the invention relates to an innovative technique for integrating an identification element in a protuberance on a surgical instrument or on another element to be identified.

The present invention aims to be resistant over time, to have a lifetime equivalent to that of the elements to be marked, it aims to be inexpensive, in order to be able to provide a maximum of elements to be marked, it aims to be easily usable: an easy, multiple and remote detection, aims to be secure, not having any asperity which could lead to the presence of infectious agent, and finally resistant to the sterilization environment.

To achieve this aim, the invention will use RFID technology, recognised by those skilled in the art as the most appropriate to respond to the questions of safety, simplicity and management of sterilisation costs. The transponder or tag comprises a microprocessor, a memory, an input analogue interface and an antenna. A transponder is connected to the element to be marked, as will be shown in the invention, in order to assure rapid identification of the appropriate element, by detection of this transponder remotely by a suitable reading system. The system is connected to an information system making it possible to recover the information items linked to the element to be marked, in certain cases via a data base. Also, the benefice of this technology are an important storage of information items, a rapid and multiple reading. The difficulty of the present invention resides in the association of the RFID tag with the element to be marked.

The benefice of the use of an RFID tag, or transponder, for the present invention are numerous, those skilled in the art will easily identify the potential of this technology coupled with the present invention, herewith a series of illustrations of these benefices, this list is non-exhaustive; the use of the invention should not be limited to the sole examples presented below. The use of an RFID tag or transponder in the present invention has, among other things, the aim of linking each patient with all of the instruments used during his operation, and each instrument to all of the patients for which it will have served, to check that an instrument has indeed undergone all of the sterilisation steps by an automatic or manual detection at each of the sterilisation steps, in the case of the non-respect of these steps, the instrument must be able to be localised and removed from the cycle, to quantify the number of sterilisation cycles and operations that the instrument will have undergone, to know its date of purchase in order to be able to use the manufacturer's guarantees. Another benefice of this technology is that it is sometimes already used in sterilisation for the detection of instrument containers, boxes or bags, which enables an adaptation of systems already in place.

In an embodiment of the invention, the transponder, or RFID tag, is integrated in a protuberance, or housing, of specific material, the housing being integrated in the element to be marked so that no part of the transponder is too close to the element to be marked. An embodiment of the present invention targets a reusable or non-reusable medical device, comprising:
a protuberance of material enabling, at least partially, electromagnetic waves to pass, integral with the medical device,
an electronic chip responding to the radio frequencies inserted entirely, or partially, in the protuberance.

In the remainder of the document, “tag” will designate any type of RFID component such as the transponder, or RFID tag, or RFID chip, or electronic chip, or radio label. The characteristic positioning of the tag outside of the element to be marked in a non-conducting material which, associated or not with an insulator, makes it possible to limit Faraday cage effects in the case of elements to be marked made from metal, which facilitates the transfer of information items remotely between the reader and the tag, this characteristic constitutes the innovation of the present invention, it is the original approach of this solution.

In an embodiment of the invention, the material is a resin. Several large families of resins may correspond to the objectives of the present invention: Epoxy type resins, Polyamides, Polycarbonates, Polystyrenes, Polyethylenes, Polypropylenes, Acrylonitriles, Butadienes, Styrenes, Acrylics, Polyurethanes, Nylons. A focus will be made, in the remainder of the document, on Epoxy type resins. This resin will be more particularly an EPOXY type resin, two component, with high thermal stability and resistant to the effects of water for plastic-metal compatible substrates, complying with medical standards. It must be obvious to those skilled in the art that the present invention enables embodiments under numerous other specific forms, using different resins for different applications, without going beyond the scope of the invention. Consequently, the present embodiments should be considered by way of illustration, and the invention should not be limited to the single resin presented below.

These two component EPOXY adhesives develop high resistance to the chemical agents used during pre-washing, but also to effects due to traction and to shear on a large variety of plastics and metals. The polymerisation of the resin-hardener couple creates a cohesive thermosetting plastic with good chemical and thermal resistance. During their integration and their use, the resin has the characteristic of having a limited shrinkage effect. Their final structure must have the characteristic of being completely homogeneous, and in particular without air bubbles, in order not to degrade their mechanical strength, but also without asperity at their surface in order to comply with the health prerogatives imposed by the supervisory authorities.

The inventor has determined that this type of particular material meets the thermal, chemical and mechanical constraints for the linking of the tag and an element to be marked and has a surface state unfavourable to depositions.

The integration of the resin in the element to be marked may be carried out according to two particular methods: one by moulding in the case of a liquid or solid resin, the other by bonding in the case of a pasty resin, to the element to be marked. The method by bonding implies a prior preparation of the resin used: the resin is pre-cut and in certain cases formed in order to receive the tag, the cut will make it possible to receive the part of marking of the element to be marked. The tag will be integrated in the resin.

According to particular embodiments, the tag may be integrated directly with the resin in the element to be marked, this integration in the element could take place either by moulding, or by bonding, or by any other method of bonding resin on metal, according to a particular method.

According to other particular embodiments, the protuberance is firstly integrated in the element to be marked, this protuberance having a hollow area of the shape of the tag with a supplement of space enabling closure, this integration of the protuberance in the element could take place either by moulding, in the case of a liquid or solid resin with a mould having the requisite shape as well as the housing required to receive the tag, or by bonding, in the case of a pasty resin, by pre-cutting of the requisite shape as well as the housing of the tag, or by any other method of bonding of resins on metal, this according to a particular method. The tag will then be positioned in the space being left vacant for it in the protuberance, then a cover, of same composition as the protuberance, will be placed on the tag in order to close the opening. The cover could be welded by ultrasounds by means of a sonotrode, to the protuberance, according to a particular method. According to another particularity, the cover is bonded to the body of the protuberance so as to assure the leak tightness of the housing. The welding or bonding of the cover makes it possible to assure continuity between the surface of the protuberance and the cover in order to guarantee the leak tightness of the housing of the tag.

According to other particular embodiments, the protuberance is firstly integrated in the element to be marked, this protuberance not having a hollow area, this integration of the protuberance in the element could take place either by moulding, in the case of a liquid or solid resin with a mould having the requisite shape, or by bonding, in the case of a pasty resin, by pre-cutting of the requisite shape, or by any other method of bonding resins on metal, this according to a particular method. A housing enabling the integration of the tag and a cover is then machined, according to a particular method, at the positioning of the tag defined on the protuberance. The tag will then be positioned in the space being left vacant for it in the protuberance, then a cover, of same composition as the protuberance, will be placed over the tag in order to close the opening. The cover could be welded by ultrasounds, using a sonotrode, to the protuberance, according to a particular method. According to another particularity, the cover is bonded to the body of the protuberance so as to assure the leak tightness of the housing. The welding or bonding of the cover makes it possible to assure continuity between the surface of the protuberance and the cover.

According to another particularity, the diameter and the depth of the housing formed by the machining are substantially greater than the diameter and the depth of the tag and of the cover so that the tag is positioned in a housing equidistant from the flat upper parts of the protuberance when the cover is welded or bonded to the body of the element to be marked.

According to another particularity, it will be advised to leave a minimum distance of at least 1 mm between the tag and the instrument, space filled by the resin, and a sufficient distance of material between the tag and the exterior in order that the latter is totally isolated in the resin protuberance, in order to assure the protection of the tag, the solidity of the protuberance, as well as limiting the perturbations caused by other metal elements located near to the protuberance during identification.

For this invention, several possibilities of tags could be proposed according to the expected applications of the invention by those skilled in the art.

An aim is attained by proposing a use of an element to be marked according to an embodiment of the invention, in this not limited embodiment the tag is a passive tag in the case where the user desires an inexpensive solution.

Another aim is attained by proposing a use of an element to be marked according to an embodiment of the invention, in this not limited embodiment the tag is an active tag, if the user wishes a solution taking into account more information items, and offering wider operating possibilities.

Another aim is attained by proposing a use of an element to be marked according to an embodiment of the invention, in this not limited embodiment the tag is a tag in simple reading mode, if only the code of the instrument and certain fixed information items interest the user.

Another aim is attained by proposing a use of an element to be marked according to an embodiment of the invention, in this not limited embodiment the tag is a tag in reading and writing mode, in the case where the user desires a solution enabling him to register the information items also collected on the memory of the tag, thus on the element to be marked.

Another aim is attained by proposing a use of an element to be marked according to an embodiment of the invention, in this not limited embodiment the tag is a low frequency tag, having few perturbations linked to metals or to the human body, but a lower reading speed, thus a more difficult multiple reading of the element to be marked.

Another aim is attained by proposing a use of an element to be marked according to an embodiment of the invention, in this not limited embodiment the tag is a high frequency or ultra high frequency tag enabling a rapid and thus multiple reading. For HF and UHF tags, the present solution makes it possible to reduce the Faraday cage effects linked to the metal of the element to be marked, by a positioning of the tag at a distance from the element to be marked.

It must be obvious to those skilled it the art that the present invention enables embodiments under numerous other specific forms, by integrating different tags for different applications, without going beyond the scope of the invention. Consequently, the present embodiments should be considered by way of illustration, and the invention should not be limited only to the tags presented above.

According to another particularity, the tag used for the present invention could be encapsulated in a structure before being integrated directly in the material. According to another particularity, the tag could be directly integrated in the material without having been enclosed beforehand in a protective structure.

In an embodiment of the invention, the resin protuberance containing the tag is integrated in the element to be marked such that its position is in no way bothersome for an optimal use by the user, or for the implementation of a handle.

According to another particularity, the shape of the protuberance could be adapted to the expectations of the medical staff, while assuming that the optimal shape remains that which we will present in the course of the explicative description, this shape having the aim of perturbing as little as possible the grip of the user.

Thanks to these devices, the link between the chip and the reusable element to be marked is, at one and the same time, very resistant to mechanical shocks, to aggressive environments, particularly to chemical cleaning agents, the resin protuberance serving as protection to the tag, and sufficiently flexible to withstand different thermal expansions of the chip and of the reusable medical device. In addition, the fact that the chip is present at a minimum distance from the metal element to be marked, the effects of perturbation of the element on the waves are reduced, the fact of using a material of EPOXY resin type means that the risks of depositions are limited, or even inexistent.

Other aims, characteristics, details and benefices of the present invention will become clearing in the course of the description that follows, description given in an explicative aim and in no way limiting, with references to the appended figures given by way of non-limiting example in which:

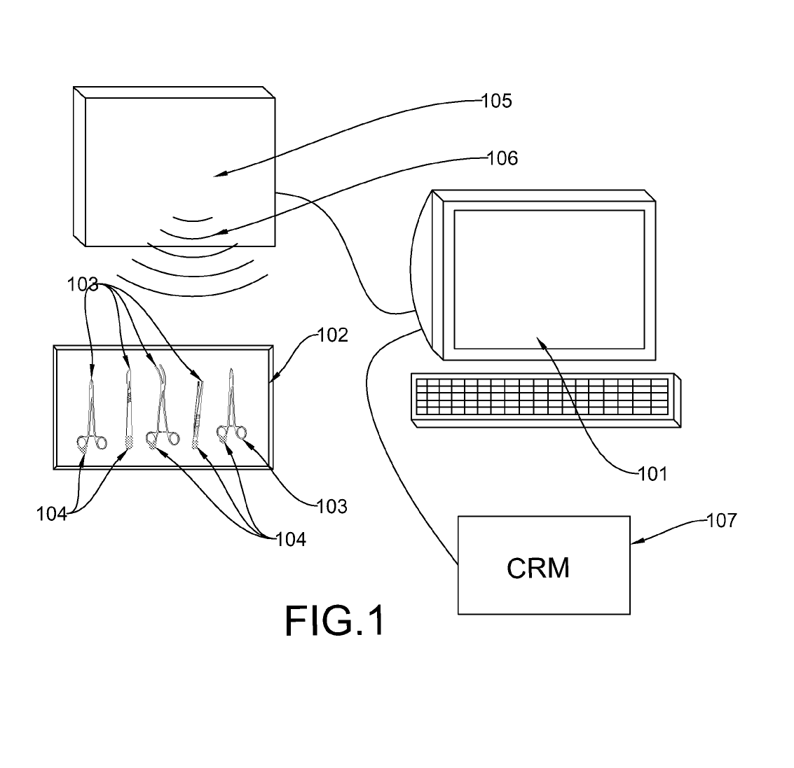

FIG. 1 illustrates the use of a surgical instrument marked by means of the present invention, cooperating with a reading system associated with an information system.

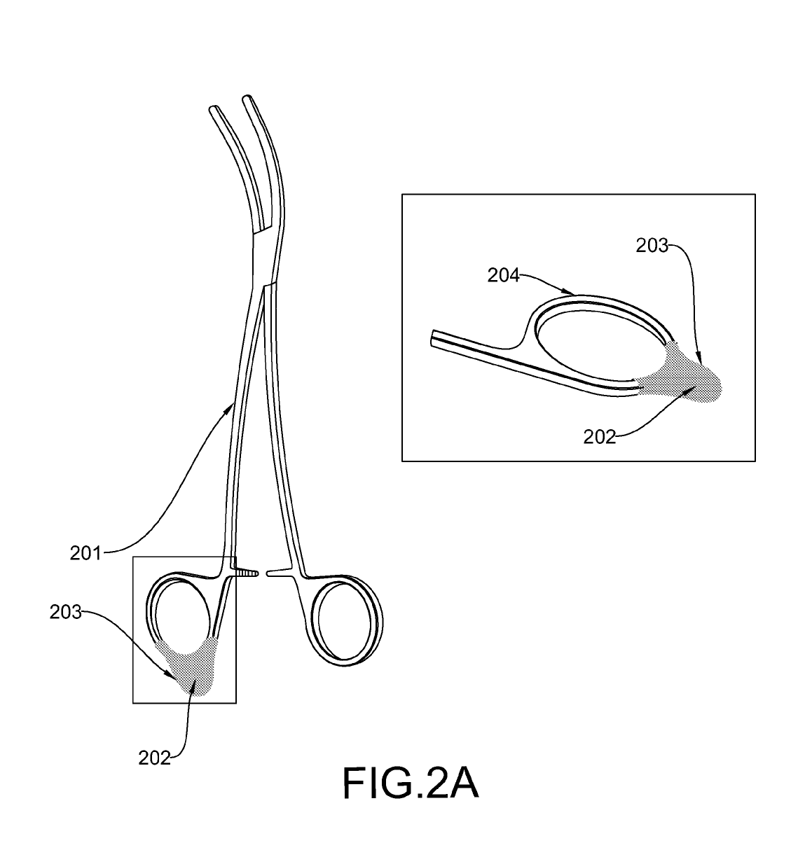

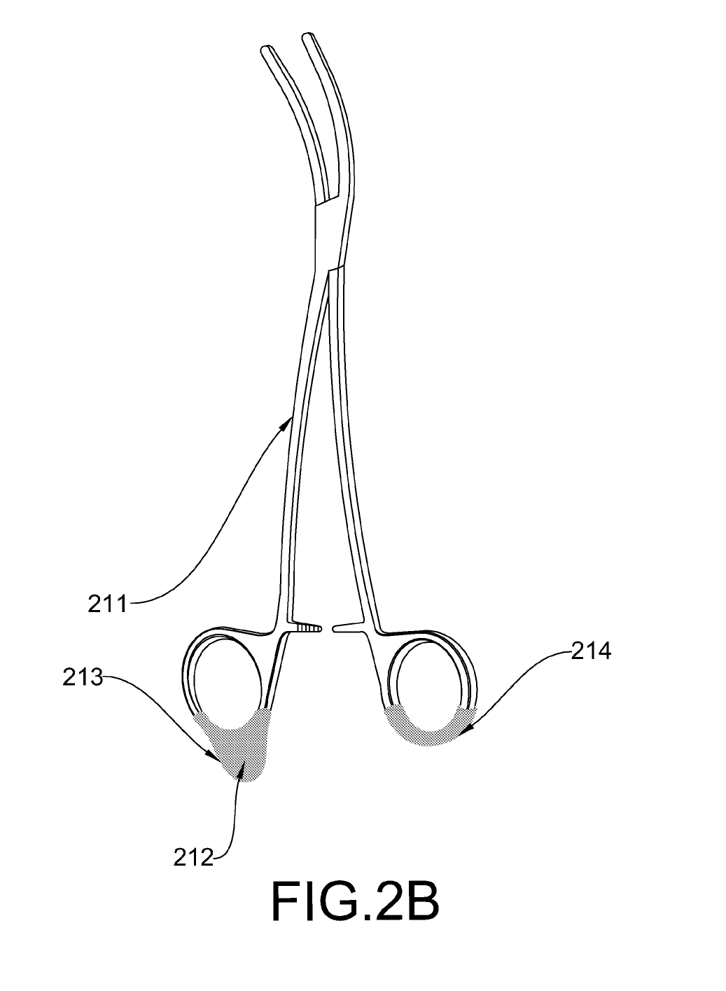

FIG. 2A represents in top view a surgical instrument, such as an iliac clamp, on which has been integrated the resin protuberance comprising the RFID component, which is the tag, on one of the rings of the scissors, as well as an enlarged top view of this marked ring.

FIG. 2B represents in top view a surgical instrument, such as an iliac clamp, on which has been integrated the resin protuberance containing the RFID component on one of the rings, as well as a protuberance of different shape on the other ring, protuberance normally not comprising an RFID component.

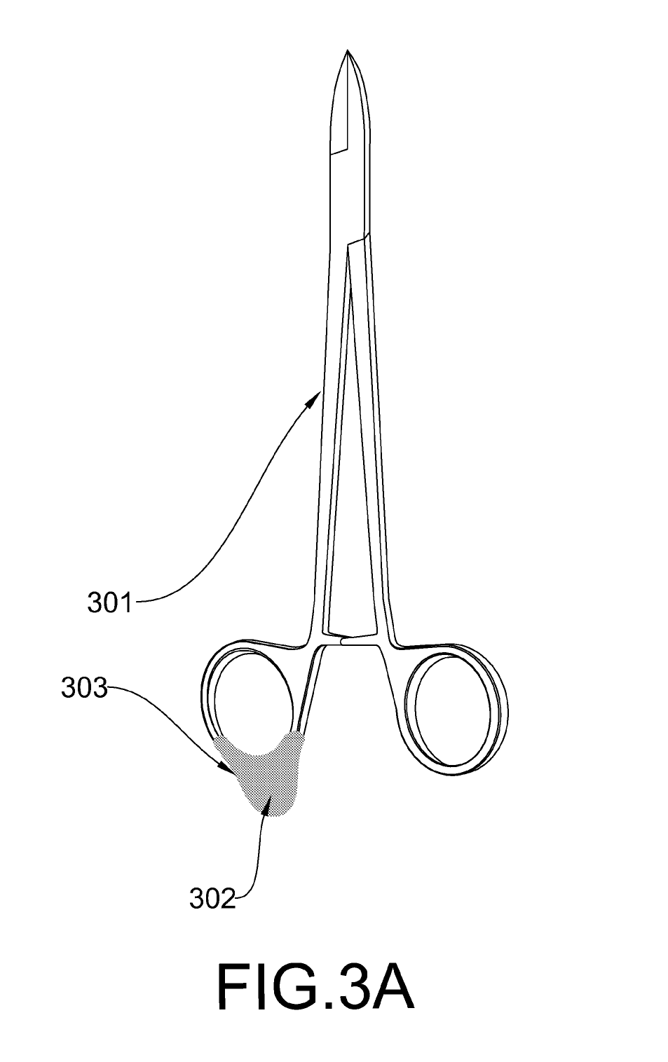

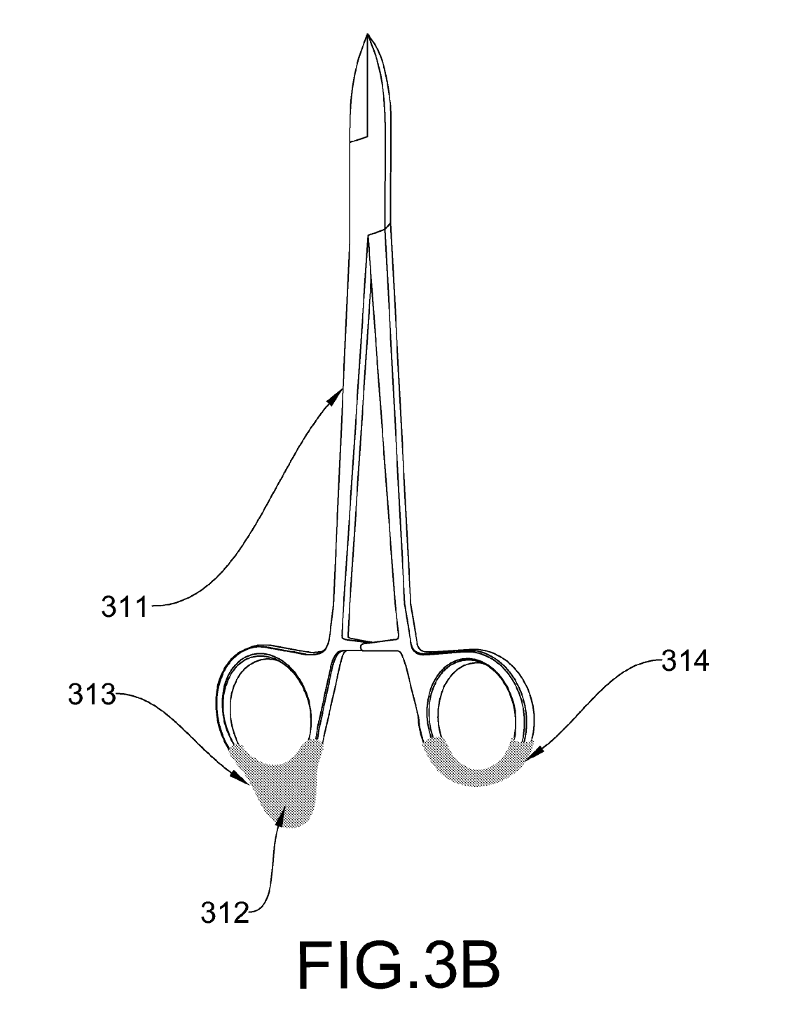

FIG. 3A represents in top view a surgical instrument, such as the needle holder, on which has been integrated the resin protuberance comprising the RFID component, which is the tag, on one of the rings of the scissors.

FIG. 3B represents in top view a surgical instrument, such as the needle holder, on which has been integrated the resin protuberance containing the RFID component on one of the rings, as well as a protuberance of different shape on the other ring, protuberance normally not comprising a RFID component.

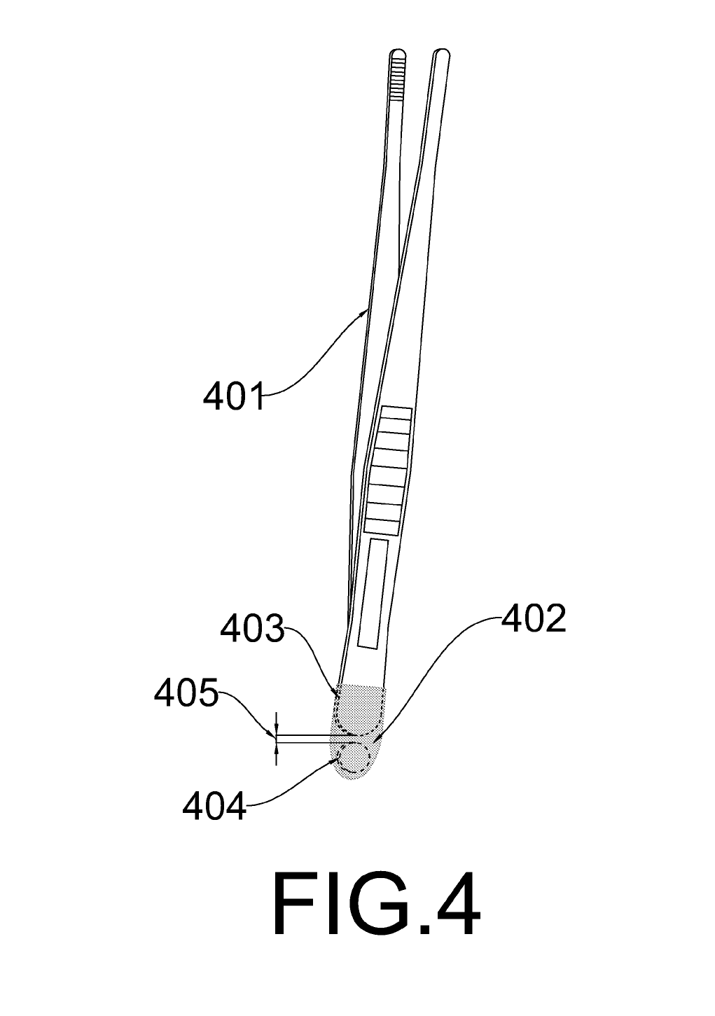

FIG. 4 represents in top view a surgical instrument, such as a clamp known as “Cushing PAD”, on which has been integrated the resin protuberance containing the RFID component, which may be seen in transparency.

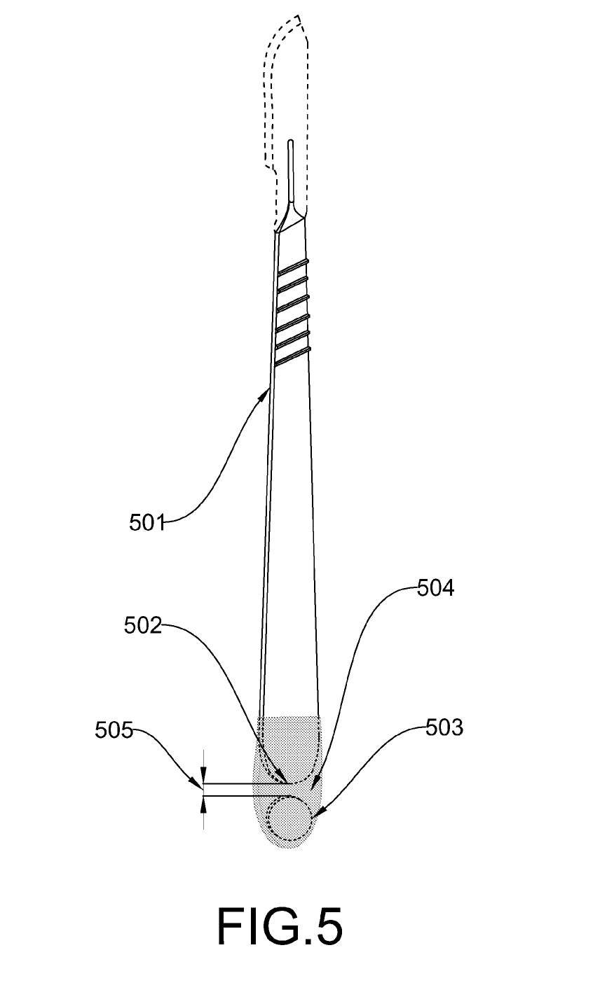

FIG. 5 represents in top view a surgical instrument, such as a scalpel, the blade having been drawn in dotted lines, the handle in solid lines, on which has been integrated the resin protuberance containing the RFID component, which may be seen in transparency.

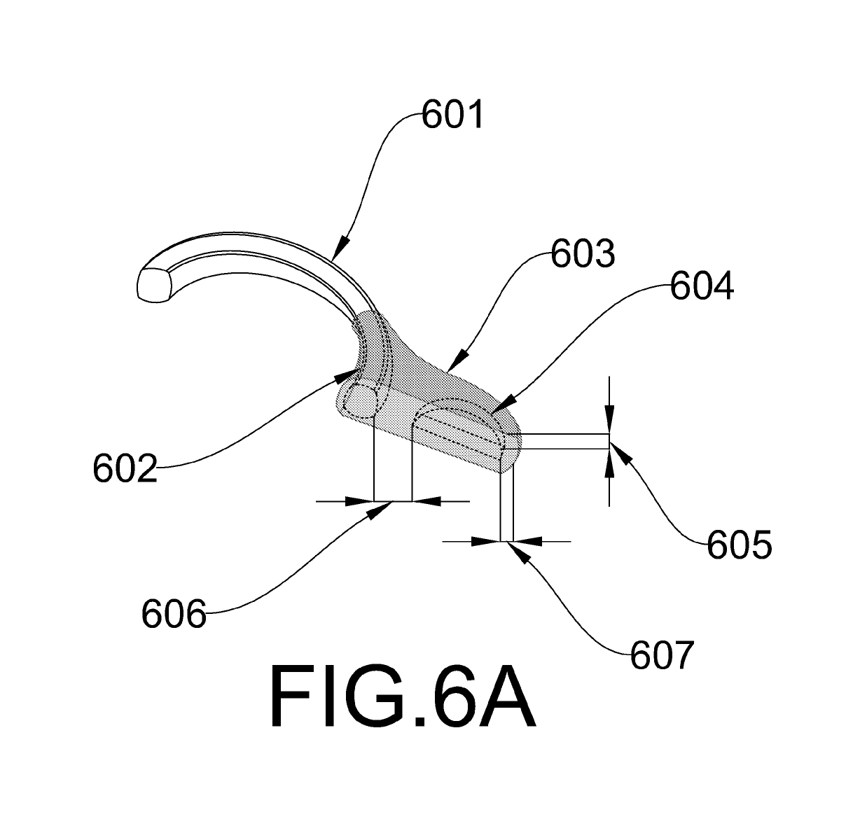

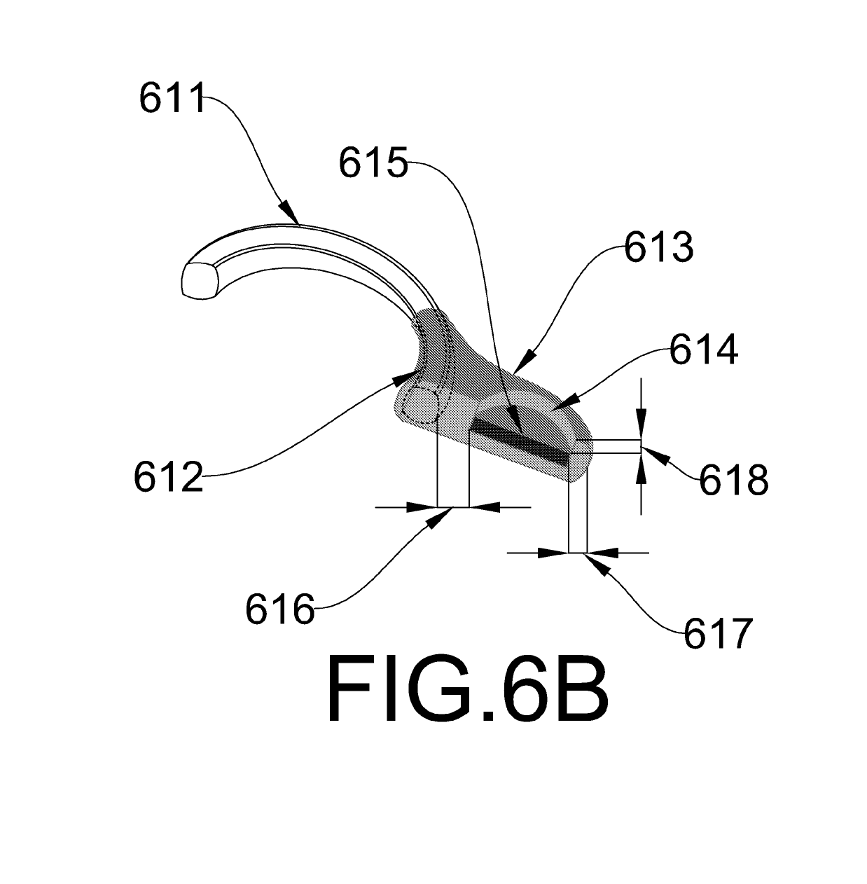

FIG. 6A is a longitudinal sectional view of marked rings of the instruments represented in FIGS. 2A, 2B, 3A, 3B, it makes it possible to visualise the interior of the protuberance, and also to see in transparency the RFID component. This figure represents a mode of integrating the protuberance and RFID component, in the instrument.

FIG. 6B is a longitudinal sectional view of marked rings of the instruments represented in FIGS. 2A, 2B, 3A, 3B, it makes it possible to visualise the interior of the protuberance, and also to see in transparency the RFID component. This figure represents another mode of integrating the protuberance, and the RFID component, in the instrument.

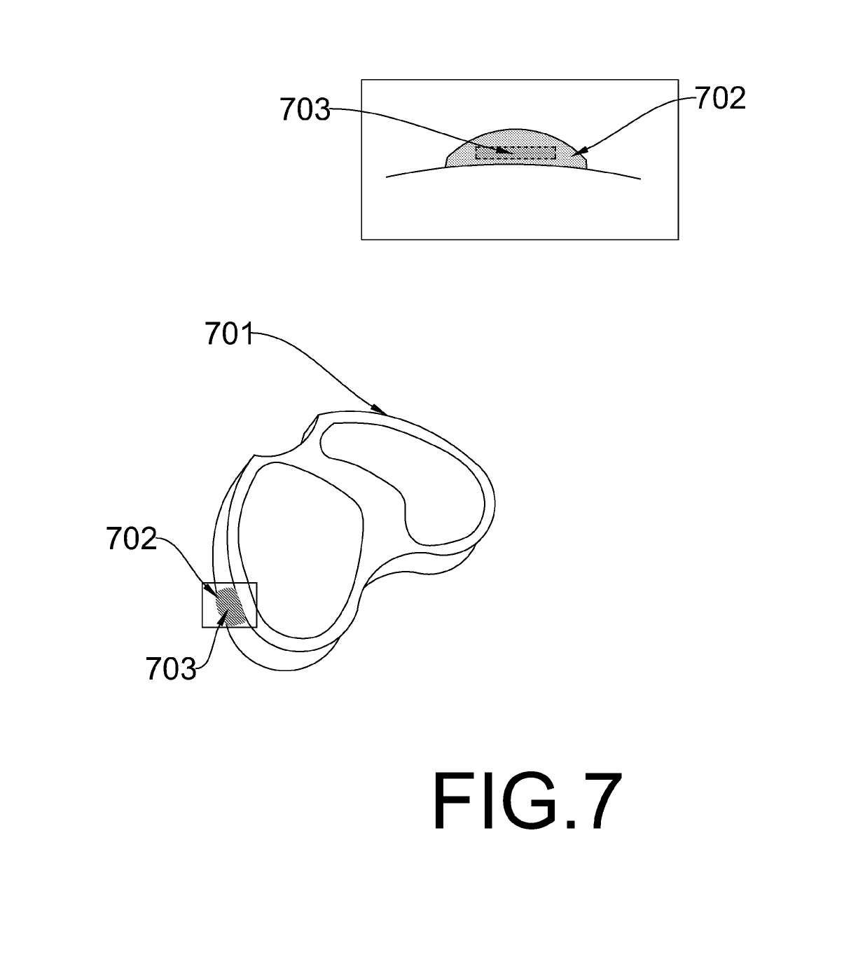

FIG. 7 represents in top view an implant or prosthesis substitute made of polymer or plastic, which is here a tibial prosthesis, on which has been integrated a resin protuberance comprising an RFID component. An enlarged sectional view of this protuberance has also been added, the RFID component may be seen therein in transparency.

Throughout the description, medical devices will be presented such as reusable metal surgical instruments, or instead a prosthesis substitute made of polymer, however, the scope of the present invention is not limited only to this type of reusable medical device.

Generally speaking, the medical device according to an embodiment of the invention comprises a reusable or non-reusable surgical instrument made from metal, polymer or plastic, any implant or prosthesis substitute made from metal, polymer or plastic, equipped with a marking system enabling rapid identification of the instrument. The marking system comprises:
an RFID component commonly called a “tag”, and
a resin protuberance rigidly connected to the surgical instrument or the implant substitute to be marked, the protuberance containing and completely surrounding the tag, the thickness of resin surrounding the tag being for example greater than or equal to 1 mm.

In a not limited embodiment, the resin of the protuberance is an EPOXY type resin.

In one embodiment, the protuberance integrating the tag is fixed on the surgical instrument or on the implant substitute to be marked in a location corresponding to a part forming means of gripping the instrument. It should be noted that the part of the protuberance coupled to the surgical instrument or the implant substitute to be marked hugs the shape of the instrument or the implant substitute.

Moreover, the medical device according to an embodiment of the invention may comprise at least one additional protuberance, the latter being adapted to balance the surgical instrument or the implant substitute. Moreover, this additional protuberance may contain a tag.

In different embodiments, at least one protuberance:
contains at least two tags, the at least two tags being oriented along different directions,
contains a radio opaque element,
is coloured according to a colour code making it possible to identify the medical device.

In one embodiment, a radiofrequency insulator is arranged between the instrument or the implant substitute to be marked and the protuberance.

The surgical instrument 103, 201, 211, 301, 311, 401, 501, 701, equipped with an RFID component 202, 212, 302, 312, 404, 503, 604, 615, 703 enabling its remote identification makes it possible to improve the monitoring of the instrument, its traceability within the establishment, as well as in certain cases its traceability between different health establishments also improves the management of the instrument, by associating with it for example the patient operated, management of stocks and inventories, this list is non-limiting.

With reference to the figures, an embodiment of the invention proposes adding on a metal, polymer or plastic surgical instrument or on an implant or prosthesis substitute 103, 201, 211, 301, 311, 401, 501, 701, a protuberance of material enabling electromagnetic waves to pass, here more particularly a resin, 104, 203, 213, 303, 313, 402, 504, 603, 613, 702, in which is located an RFID component 202, 212, 302, 312, 404, 503, 604, 615, 703. In the remainder of the description, the term “surgical instrument” should be taken in a non-limiting manner. This term can encompass reusable or non-reusable surgical instruments, made from metal, polymer or plastic, or an instrument in part of one, implant or prosthesis substitutes, dental or veterinary instruments. The instruments presented in the diagrams are to be taken as examples.

In the remainder of the description, tag will be spoken of in a non-limiting manner, the term “tag” will encompass any type of RFID component. These RFID components could be encapsulated beforehand in a protective structure, or could be bare tags. For reasons of clarity, one will take as basis for the illustrations a circular tag of conventional dimensions, of diameter of one to two millimetres, and of a height of around a half millimetre. The present descriptions and examples proposed should be considered by way of illustration, but may be modified by the expectations of the user, the invention should not be limited to the details given above.

The surgical instrument presented in the example of FIG. 2A is an iliac clamp 201, which is an instrument making it possible for example to enlarge a natural cavity to facilitate the examination thereof and potentially to introduce therein an instrument.

In this example, the location of the resin protuberance 203 is provided on one of the two rings of the scissors 204. The tag 202 being located enclosed inside this protuberance.

FIG. 3A represents a second example of surgical instrument marked by means of the present invention, the instrument illustrated is a needle holder 301 which is an instrument used by surgeons for handling more easily suture needles. In this example, the location of the resin protuberance 303 is provided on one of the two rings of the scissors. The tag 302 is located enclosed inside this protuberance.

In these examples, the protuberance 203, 303 has an elliptical part in which is located the tag 202, 302, the part coupled to the ring 204 hugs the shape of the ring 204, in order not to perturb the grip of the scissors 201, 301 by the surgeon. The half-ellipse shape containing the tag 202, 302 also makes it possible to perturb as little as possible the grip of the instrument 201, 301 by the surgeon, this shape above all makes it possible to hug the shape of the instrument and the tag in order to limit the size of the protuberance, but also to limit its weight. A benefice of this invention is that the positioning of the tag within the protuberance is such that the perturbations on the transmissions of information items by waves, due to metal instruments, are limited. In addition, the structure of the instrument is not modified, the instrument is not machined, which limits the marking costs, as well as the risks of weakening the structure of the instrument.

It should be noted that the protuberance 203, 303 is arranged in the continuity of the instrument to be marked such that the junction between the protuberance 203, 303 and the instrument to be marked does not have any uncoupling (flaw), such a junction precluding the deposition (the nesting) of bacteria between the protuberance 203, 303 and the instrument to be marked.

In a variant, the protuberance could be located on another portion of the instrument and have a different shape, this will depend on the expectations of the user, the present invention is not limited to the applications proposed by the present description, a protuberance of different shape could be placed on the ring, or on the junction area between the two parts of the ring; nevertheless, this type of instrument being invasive, it is then necessary that the part of the instrument in contact with the patient retains its initial shape, the protuberance must thus not be located on these parts. The benefices of the positioning, and of the shape proposed in this example, are that the shape hugs the shape of the ring, without leading to considerable perturbation for the surgeon.

The protuberance 203, 303 will be, for example, made from EPOXY type resin, with high thermal stability for plastic metal substrates compatible with medical standards, these EPOXY components develop a high resistance to pre-washing, to traction and to shear on a large variety of plastics and metals. They polymerise to form a cohesive thermo-setting plastic with good thermal and chemical resistances, they result in low shrinkage. This type of particular material meets the thermal, chemical and mechanical constraints for the linking of the tag and a reusable surgical instrument and has a surface state unfavourable to depositions. This resin could be integrated in liquid, solid form, or in the form of paste.

The use of a resin as material confers on the protuberance 203, 303 a high resistance to the aggressive conditions of sterilisation cycles.

In an embodiment of the invention, the resin used for the protuberance 203, 303 is biocompatible.

Passive tags, here cylindrical 202, 302, are known to those skilled in the art for their use for markings of objects, these tags are composed of an electric circuit with two sheets on which are welded a coil formed of a wire wound round a ferrite core.

The present invention proposes a use of surgical instruments marked as described in FIG. 1. The surgical instruments 103 marked by means of the present invention 104 are here grouped together on a tray 102, this description being an illustration of the use of the invention that is non-limiting. The reading/writing of the tags present in the protuberances 104 of the surgical instruments 103 is carried out by means of the antenna of the tag formed of the annular coil able to transmit and receive a radiofrequency signal containing information items, a computer software 101 will make it possible to ensure the interface between the user and the data stored on the tags, as well as on a data base 107 coupled automatically to the detection of marked surgical instruments. A transmitter/receiver 105 will make it possible to ensure communication between the instruments and the software, the software establishing the link between the data base 107 and the user. This transmitter/receiver 105 transmits an electromagnetic field 106 containing the information, it is this field 106 that will make it possible to bridge information items between the tag of the surgical instrument 103 and the software 101.

The use of the present invention as marking of a surgical instrument has in its use a step of reading of information items contained in the tag situated in the protuberance 104 by means of the reading system 105, the transmission of the information items to the software that forms the interface with the user 101 and the data base 107, a step of transmission of data by the software to the user, as well as an updating of the data base 107. The traceability data linked to the surgical instrument, stored in the database 107, can be modified as identifications are made of the surgical instrument 103, in an automatic manner by the software or manually by the user, these functions will be adapted as it suits the user.

Other examples of marking by means of the present invention are presented in FIGS. 4 and 5. FIG. 4 presents a surgical instrument 401, more precisely a “Cushing” clamp 401 aiding the surgeon in dissection, FIG. 5 presents as surgical instrument a scalpel 501. In this example, the location of the resin protuberance 402, 504 is provided at the bottom of the surgical instruments, in the continuity of the surgical instruments. The tag 404, 503 is located enclosed inside this protuberance.

In these examples, the protuberance 402, 504 has an elliptical part in which is located the tag 404, 503, the part coupled to the lower part of the surgical instrument 403, 502 hugs the shape of the surgical instrument 403, 502 so as not to perturb the gripping of the surgical instruments 401, 501 by the surgeon. The semi-ellipse shape containing the tag 404, 503 also makes it possible to perturb as little as possible the gripping of the surgical instrument 401, 501 by the surgeon, this shapes makes it possible above all to hug the shape of the surgical instrument 403 and the tag in order to limit the size of the protuberance, but also to limit its weight. Another benefice of this solution is the structure of the surgical instrument 401, 501 is not modified, the surgical instrument 401, 501 is not machined, which limits the costs of marking, as well as the risks of weakening the structure of the surgical instrument 401, 501.

In a variant, the protuberance could be located on another portion of the surgical instrument and have a different shape, this will depend on the expectations of the user, the present invention is not limited to the applications proposed by the present description, a protuberance of different shape could be placed on the lower part of the surgical instrument, or on another area of the instrument, but it should not be forgotten that this type of surgical instrument is invasive, it is then necessary that the part of the surgical instrument in contact with the patient retains its initial shape, the protuberance should thus not be located on these parts. The benefices of the positioning, and of the shape proposed in these examples, are that the shape hugs the shape of the ring, without leading to a significant perturbation for the surgeon.

It will be noted that the transparency view present in these two figures makes it possible to see the positioning of the tag 404, 503 with respect to the surgical instrument 401, 501 inside the resin 402, 504. It will be noted that a minimum distance 405 has to be left between the chip and the structure of the surgical instrument. In fact, the aim of the present invention being among others to limit the perturbations caused on the transfer of information items between the tag and the transmitter/receiver, the positioning of the tag is then primordial, a distance of at least 1 to 2 mm must be left between the surgical instrument and the tag, the interstice being filled by the resin.

In a variant of FIGS. 2A and 3B, which present two solutions of integrations of the present invention to two examples of surgical instruments having a base composed of two rings, it is possible to integrate on the second ring a second resin protuberance, this second protuberance being able to serve to balance the surgical instrument to thereby not modify the working habits of the surgeon.

In a variant, this second protuberance could also contain a tag, in order to increase the field of detection of the surgical instrument to be marked. It could also be imagined to integrate several tags in each of the protuberances, tags oriented in different directions.

In a variant, it could be possible to multiply the resin protuberances on an instrument, each of the protuberances being able to contain one or more tags, this would make it possible to multiply the chances of identification of the marked surgical instrument. These tags could be positioned in different directions, in order to increase the chances of identification of the surgical instrument.

In a variant, the resin of the protuberances could be coloured, according to a predefined colour code, which would make it possible to facilitate the visual traceability of the instruments, when there is no reading systems. In fact, a colour code adapted to the operating theatre number, or to the surgeon, could be adopted according to the health establishment in which the traceability system is installed.

In a variant, to the resin of the protuberance could be added a radio opaque element, easily localisable by radio waves, enabling a simple localisation, this would make it possible to verify easily in the event of loss of an instrument in the operating room that the latter has not remained in the body of the patient.

In a variant, an objective is attained when for each of the methods mentioned previously, an insulator could be applied at the interface between the resin protuberance and the marking area 602, 612 of the metal of the instrument to be marked. A step of integration of the insulator could then be added in the methods described previously, before the step of integration of the resin protuberance in the instrument.

FIG. 6A presents a sectional view of the instruments 601 presented in FIGS. 2 and 3, this figure makes it possible to understand a method of integration of the protuberance 603. It may be noted in this figure that the resin protuberance 603 closely hugs the shape of the ring 602 of the instrument. This figure illustrates the following method of integrations of the protuberance 603, according to particular embodiments the tag 604 may be integrated directly with the resin in the instrument to be marked 601. During this step, an insulator could be added at the level of the resin-metal interface 602. This integration in the surgical instrument could take place by moulding in the case of a liquid or solid resin, under vacuum or not, a mould hugging the shape of the ring 602 could be used. The mould will have to respect precisely the position of the tag 604, in fact as explained previously the position of the tag with respect to the instrument is very important to limit the perturbations of the metal surgical instrument on the electromagnetic field enabling transfers of information items between the tag and the identification system, a minimum distance 606 of at least 1 mm must be respected between the instrument 602 and the tag 604. In addition, a sufficient quantity of resin must surround 605, 607 the tag, a height of resin of at least 1 mm will be respected, this thickness of resin will make it possible not to weaken the structure, and a multiple reading in the case where the marked surgical instrument is surrounded by metal instruments.

In a variant, the method of integration of the resin protuberance 603, tag 604 couple in the instrument could take place either by bonding in the case of a pasty resin, the method by bonding implies a preparation beforehand of the resin used: the resin is pre-cut and formed in order to receive the tag 604, the cutting will make it possible to receive the marking part of the element to be marked 601. The tag 604 will be directly integrated in the space that has been left vacant for it. The couple formed of resin protuberance 603, tag 604, will be integrated in the surgical instrument 601 by bonding. During this step, an insulator could be added at the level of the resin-metal interface 602.

FIG. 6B presents a sectional view of the instruments 611 presented in FIGS. 2 and 3, this figure makes it possible to understand a method of integration of the protuberance 613. It may be noted in this figure that the resin protuberance 613 closely hugs the shape of the ring 612 of the instrument. This figure illustrates the following methods of integration of the protuberance 613, according to particular embodiments the protuberance 613 is firstly integrated in the element to be marked. During this step, an insulator could be added at the level of the resin-metal interface 612. This protuberance 613 has a hollow area 615, 614 of the shape of the tag with an additional space enabling closure 614. This integration of the protuberance 613 in the surgical instrument 611 could take place either by moulding, under vacuum or not, by means of a mould hugging the shape of the ring as has been described previously. The hollow must respect precisely the position of the tag 615, in fact as explained previously, the position of the tag with respect to the instrument is very important to limit the perturbations of the instrument on the electromagnetic field enabling transfers of information items between the tag and the identification system, a minimum distance 616 of at least 1 mm must be respected between the instrument 612 and the tag 615. In addition, a sufficient quantity of resin will have to surround 617, 618 the tag, a height of resin of at least 1 mm will be respected, this thickness of resin will make it possible not to weaken the structure, and a multiple reading in the case where the marked surgical instrument is surrounded by metal instruments. The diameter of the hollow will be substantially greater than the diameter of the tag 615. The depth of the hollow will be substantially greater than the sum of the depth of the tag 615, and the height of resin required 618 to cover the tag 615 in its cavity, in order to assure a protection, and a maximum resistance of the resin 613, tag 615 couple.

In a variant, the method of integrating the resin protuberance having a hollow as described previously 614, 615, could take place either by bonding in the case of a pasty resin, the method by bonding implies a preparation beforehand of the resin used: the resin is pre-cut according to the desired shape 613, leaving the defined housing for the tag 615 according to the characteristics in the preceding paragraph, or by any other method of bonding resin on metal, according to a particular method, under vacuum or not. During this step, an insulator could be added at the level of the resin-metal interface 612. The couple formed, resin protuberance 613, tag 615, will be integrated in the surgical instrument 601 by bonding.

The tag 615 will then be positioned or wedged in the housing 615 left vacant for it in the protuberance 613. The method of integrating the tag 615 in the instrument then comprises a step of positioning the cover 614, the cover will be of same composition as the protuberance 613. The cover will be placed on the tag in order to close the opening. Then a step comprising an operation of welding of the cover to the remainder of the protuberance 613 will be carried out in order that the external surface of the protuberance does not have any projecting asperity. The welding could take place by ultrasounds by means of a sonotrode, according to a particular method.

In a variant, the cover 614 is bonded to the body of the protuberance 613 so as to assure the leak tightness of the housing. The welding or bonding of the cover, under vacuum or not, makes it possible to assure continuity between the surface of the protuberance 613 and the cover 614, in order to assure the leak tightness of the housing of the tag 615.

In a variant, another method of integration of the protuberance 613, tag 615 couple could be illustrated by FIG. 6B. According to particular embodiments, the protuberance 613 is firstly integrated in the element to be marked, this protuberance 613 not having any hollow area. This integration of the protuberance 613 in the element could take place either by moulding, under vacuum or not, by means of a mould hugging the shape of the ring as has been described previously.

In a variant, the method of integrating the resin protuberance 613 could take place either by bonding in the case of a pasty resin, the method by bonding involves a prior preparation of the resin used: the resin is pre-cut according to the desired shape 613, or by any other method of bonding resin on metal, according to a particular method, under vacuum or not. During this step, an insulator could be added at the level the resin-metal interface 612.

The method of integrating the tag on the instrument then comprises a step of machining the resin 613. A housing enabling the integration of the tag 615 and a cover 614 is then machined according to a particular method. The housing must respect precisely the position of the tag 615, in fact as explained previously, the position of the tag with respect to the instrument is very important to limit perturbations of the instrument on the electromagnetic field enabling the transfer of information items between the tag and the identification systems, a minimum distance 616 of at least 1 mm will have to be respected between the instrument 612 and the tag 615. In addition, a sufficient quantity of resin must surround 617, 618 the tag so as not to weaken the structure. The diameter of the housing will be substantially greater than the diameter of the tag 615. The depth of the housing will be substantially greater than the sum of the depth of the tag 615 and of the height of resin required 618 to cover the tag 615 in its cavity, in order to assure a protection, and a maximum resistance of the resin, tag couple.

The tag 615 will then be positioned or wedged in the housing 615 being left vacant for it in the protuberance. The method of integrating the tag in the instrument then comprises a step of positioning the cover 614, the cover will be of same composition as the protuberance 613. The cover will be placed on the tag in order to close the opening. Then a step comprising an operation of welding of the cover to the rest of the protuberance 613 will be carried out in order that the external surface of the protuberance does not have any projecting asperity. The welding could take place by ultrasounds by means of a sonotrode, according to a particular method.

In a variant, the cover 614 is bonded to the body of the protuberance 613 so as to assure the leak tightness of the housing. The welding or bonding of the cover makes it possible to assure continuity between the surface of the protuberance and the cover, in order to assure the leak tightness of the housing of the tag 615.

FIG. 7 presents an implant or prosthesis substitute 701 made of polymer or plastic, which is here a tibial prosthesis, on which has been integrated a resin protuberance 702 comprising a tag 703. This figure makes it possible to illustrate the method of integration of the present invention in any non-metal instrument. The method of integrating the tag 703 in the instrument comprises a step of integration of the tag 703 in its resin envelope 702. In this case again this step could take place by moulding, the mould adopting the shape of the instrument to be marked, care will be taken to respect the heights of resin to apply around the tag 703 in order to assure a good resistance of the protuberance couple, and a multiple reading in the case where the marked instrument 701 will be surrounded with metal instruments.

The method of integrating the tag 703 in the instrument 701 then comprises a step of welding the resin 702, tag 703 couple thereby moulded, this step of welding could take place by welding by ultrasounds between the polymer or plastic of the element to be marked 701 and the resin of the protuberance 702. In a variant, this step may be a step of bonding, or any other method of bonding resin on polymer, according to a particular method.

In a variant, the method of integration of the present invention on medical instruments of implant or prosthesis substitute 701 type made of polymer or plastic can also be carried out in the following manner, firstly under vacuum or not, a step of integration of a resin protuberance 702 in the implant substitute 701, the protuberance 702, either is formed beforehand by means of a mould in the case of liquid or solid resin, the mould respecting the constraints of sizes, and shape, and non-perturbation of gripping of the instrument, or formed by a cutting in the case of a use of a resin in paste form. Then carrying out of a machining of the protuberance 702, in order to create a housing for the tag 703, as well as a space making it possible to position a cover in order to enclose the housing, the cover being of the same composition as the protuberance 613, this machining will respect the positioning of the tag 703 in the resin 702, care will be taken to respect the heights of resin to apply around the tag 703 in order to assure a good resistance of the protuberance couple, and a multiple reading in the case where the marked instrument will be located surrounded by metal instruments.

Care will then be taken to position the tag 703, in the housing, the positioning of the tag having to respect the shape of the space that has been attributed to it. The cover will be placed on the tag in order to close the opening. Then a step comprising an operation of welding the cover to the rest of the protuberance 702 will be carried out in order that the external surface of the protuberance 702 has no projecting asperity. The welding could take place by ultrasounds by means of a sonotrode, according to a particular method.

In a variant, the tag may be of different shape to that presented here, it then suffices to adapt the shape of the protuberance to the tag, and thus to adapt each of the steps of the integration of the resin, tag couple to the new data.

It must be obvious to those skilled in the art that the present invention enables embodiments under numerous other specific forms without going beyond the scope of the invention as claimed. Consequently, the present embodiments should be considered by way of illustration, but may be modified in the field defined by the scope of the appended claims and the invention should not be limited to the details given above.

1. Medical device, comprising a reusable or non-reusable surgical instrument made from metal, polymer or plastic, or any implant or prosthesis substitute made from metal, polymer or plastic, which is equipped with a marking system enabling rapid identification of the instrument, implant or prosthesis substitute, wherein the marking system comprises:
an RFID component, and
a resin protuberance rigidly connected to the surgical instrument or the implant or prosthesis substitute to be marked, said protuberance containing and completely surrounding said tag.

2. Medical device according to claim 1, wherein the resin of the protuberance is an EPOXY type resin.

3. Medical device according to claim 1, wherein the protuberance integrating the tag is fixed on the surgical instrument or on the implant or prosthesis substitute to be marked in a location corresponding to a part forming a means of gripping the instrument.

4. Medical device according to claim 1, wherein the thickness of resin surrounding the tag is greater than or equal to 1 mm.

5. Medical device according to claim 1, wherein the part of the protuberance coupled to the surgical instrument or the implant or prosthesis substitute to be marked hugs the shape of said instrument or said implant substitute.

6. Medical device according to claim 1, comprising an additional protuberance.

7. Medical device according to claim 6, wherein the additional protuberance is adapted to balance the surgical instrument or the implant or prosthesis substitute.

8. Medical device according to claim 6, wherein the additional protuberance contains a tag.

9. Medical device according to claim 1, wherein a protuberance contains two tags, said two tags being oriented along different directions.

10. Medical device according to claim 1, wherein a protuberance contains a radio-opaque element.

11. Medical device according to claim 1, wherein a radiofrequency insulator is arranged between the instrument or the implant or prosthesis substitute to be marked and the protuberance.

12. Medical device according to claim 1, wherein a protuberance is coloured according to a colour code making it possible to identify said medical device.
